(19) United States
(12) Patent Application Publication
(10) Pub. No.: US 2023/0117128 A1
(43) Pub. Date: Apr. 20, 2023

(54) HOT AIR RESPIRATORY THERAPY SYSTEMS AND METHODS

(71) Applicant: Lee Kunken, Sebastopol, CA (US)

(72) Inventor: Lee Kunken, Sebastopol, CA (US)

(21) Appl. No.: 17/937,037

(22) Filed: Sep. 30, 2022

Related U.S. Application Data
(60) Provisional application No. 63/253,035, filed on Oct. 6, 2021.

(51) Int. Cl. A61M 16/10; A61M 16/00

(52) U.S. Cl. CPC A61M 16/1075; A61M 16/0066; A61M 16/06

(57) ABSTRACT

Systems and related methods for delivering hot air to a respiratory tract of a user. The system includes a hot air source comprising a heater and a blower configured to produce a flow of hot air have a temperature of at least 150° F. The flow of hot air may have a relative humidity of less than 50-20%. The system also includes a user interface connected to the hot air source by a breathing tube. The user interface is configured to receive the flow of hot air from the hot air source and deliver the flow of hot air to the respiratory tract of the user.

### BACKGROUND

### Field

The present disclosure relates to respiratory therapy. In particular, the present disclosure relates to systems and methods for delivery of hot air to the respiratory system of a user.

### Description of the Related Art

Many people use exposure to elevated heat environments, such as that provided by a sauna, in an effort to improve health. However, obtaining access to a sauna can be difficult for some people. For example, home saunas can be expensive and visiting a sauna at a gym or spa on a regular basis can be both expensive and inconvenient.

### SUMMARY

The systems, methods and devices described herein have innovative aspects, no single one of which is indispensable or solely responsible for their desirable attributes. Without limiting the scope of the claims, some of the advantageous features will now be summarized.

An aspect of the present disclosure involves a system that delivers hot air to the respiratory system of a user to at least partially simulate usage of a sauna in a more convenient and less expensive form.

An aspect of the present disclosure involves a system for delivering hot air to a respiratory tract of a user. The system includes a hot air source comprising a heater and a blower configured to produce a flow of hot air have a temperature of at least 150° F. and a relative humidity of less than 20%. The system also includes a user interface connected to the hot air source by a breathing tube. The user interface is configured to receive the flow of hot air from the hot air source and deliver the flow of hot air to the respiratory tract of the user.

In some configurations, the user interface is a nasal cannula, a mask, or a hood.

In some configurations, the hot air source includes one or both of a pressure regulator or a flow regulator.

In some configurations, the hot air source includes a temperature controller configured to adjust a temperature of the flow of hot air.

In some configurations, the hot air source comprises one or more inlet or outlet ports configured to communicate with the flow of hot air.

In some configurations, one of the one or more inlet ports is configured to permit medication to be delivered to the user via the flow of hot air.

An aspect of the present disclosure involves a method of respiratory therapy including delivering a flow of hot air to a respiratory tract of a user via a user interface. The flow of hot air has a temperature of at least 150° F. and a relative humidity of less than 20%.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through the use of the accompanying drawings.

FIG. 1 is a schematic view of an embodiment of a hot air respiratory therapy system for delivering hot air to the respiratory system of a user.

FIG. 2 is a view of another hot air respiratory therapy system.

FIG. 3 is a block diagram of an example method of use of the systems of FIGS. 1 and 2.

### DETAILED DESCRIPTION

Embodiments of systems, components and methods of assembly and manufacture will now be described with reference to the accompanying Figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

The present disclosure involves systems and related methods for delivering heated air to the respiratory system, including the lungs, of a user. The heated air can be used to simulate the environment of a sauna to provide some or all of the health or other benefits of sauna use at a lower cost and/or with greater convenience. Research suggests that the use of a sauna may speed recovery from colds and/or reduce their occurrence, possibly by weakening or killing cold viruses. The use of a sauna may also weaken or kill flu and/or other types of viruses.

The temperature range of the heated air can be between about 150 and 190° F. but in some cases can be higher, such as up to about 212 or 220° F. In some configurations, the heated air can be dry. Dry air can be air with less than about 50%, less than about 40% relative humidity or between about 20-50% relative humidity. In some instances, dry air can be air with less than about 20%, less than about 15%, or less than about 10% relative humidity, or air having a relative humidity between about 5-10%. The humidity of the heated air can be less than a humidity of the ambient air, and can fall within the desired humidity range, because of heating the air. Alternatively, the system can be configured to remove excess water vapor from the ambient air to reach the desired humidity range. Water vapor may be removed, for example, by absorption using a suitable absorbent material or adsorption, or surface hydration, using a suitable adsorbent material.

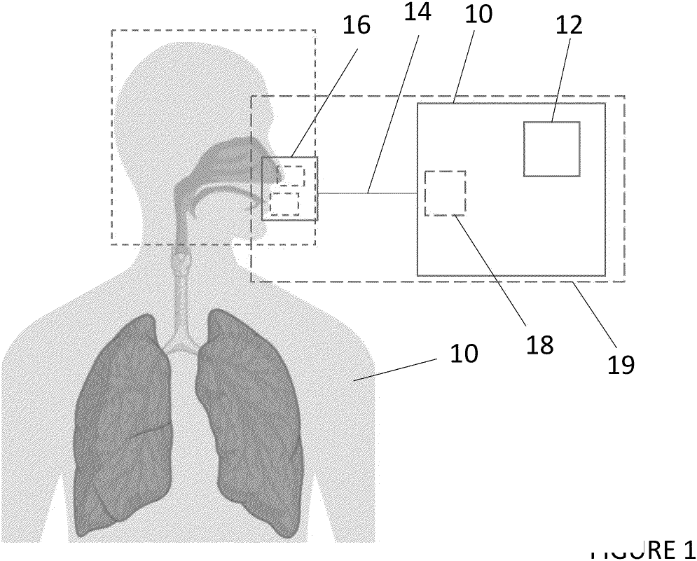

With reference to FIG. 1, in an embodiment the system can comprise a hot air source 10 configured to heat air to a desired temperature, such as up to about 200° F. or higher, as described above. The hot air source 10 can be an “instant” heat source configured to heat ambient air at least to the desired temperature at a given flow rate, which can be the rate of inhalation by the user U. In other configurations, the hot air source 10 can be configured to heat a reservoir of air (which can be refilled with ambient air). The reservoir of air can have a desired volume, which can be sufficient to meet the demands of a normal breath of a user. In some configurations, the volume of the reservoir can be sufficient to accommodate multiple breaths of a user. The hot air source 10 can be a combination of instant heating and a reservoir to reduce the demand on the instant heater. The hot air source 10 can be an electric heat source, such as one comprising one or more heating elements. The heating elements can be a metal heater wire or a ceramic element, such as a positive temperature coefficient (PTC) heater. The electric heat source can be an electromagnetic induction heater. The air can be pressurized or provided at ambient pressure. A timer may be built into the heater/blower, which can determine the period of use.

In some configurations, the hot air source 10 may include adjustable controls 12, which allows a user to adjust characteristics of the delivered air, such as temperature and/or humidity, and/or the length of the therapy session. The system can also comprise an interface portion configured to deliver the heated air to the user. The interface portion can comprise a tube or tubing 14. At the end of the tube or tubing 14 can be a hood 16 that would cover the whole head, a mask 16, nasal cannula 16 or other suitable interface that would cover or otherwise communicate with the nose and/or mouth of the user. The system will allow a user to inhale the heated air over a desired period of time, which can be between about 5-15 minutes or more, for example and without limitation.

In some configurations, the hot air source 10 or another portion of the system can include a filter 18, such as a HEPA (high efficiency particulate air) filter. The filter 18 can be integrated into the hot air source 10 or could be a separate component, such as inline with the tube 14 or integrated into the interface 16.

In some configurations, the system of FIG. 1 can be integrated into a single structure, such as include an outer housing 19, which in some cases may be comprised of a number of separate housing elements connected to one another. Thus, the schematic illustration of FIG. 1 shows the individual portions of the system separately for the purpose of descriptive convenience and understanding of the different portions of the system. However, it is intended that the illustrated of FIG. 1 depicts both a system comprised of different components and a system in which the components are integrated into a single whole. In some such configurations, the system may be incorporated in a relatively small package, which can be portable in some cases. For example, the system can be embodied in a construction similar to an electronic cigarette or vape pen utilizing the heating element to heat air instead of a liquid. If necessary, such a system can be greater in size compared to an electronic cigarette or vape pen to have sufficient power to heat the air to a desired temperature and/or to have the air exposed to the heater for a sufficient time to allow heating to a desired temperature. However, such a system can still be portable or at least relatively convenient to use and store.

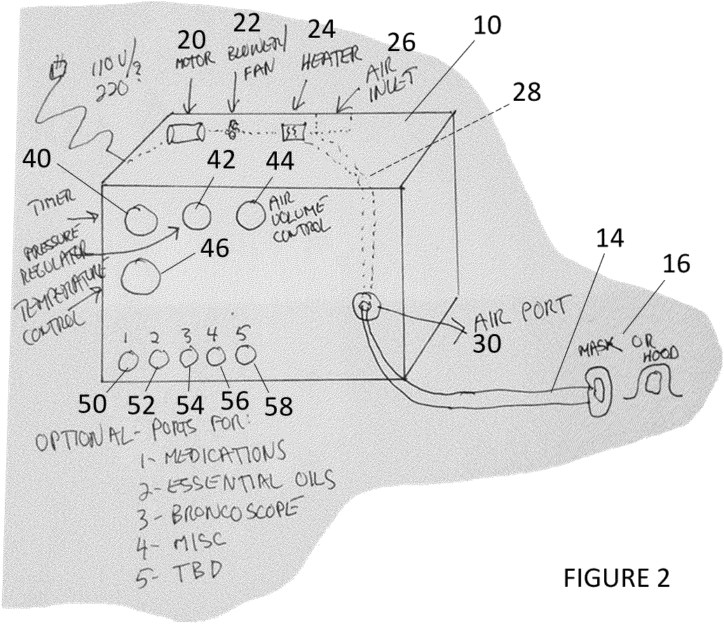

FIG. 2 illustrates another system with another hot air source 10. The illustrated hot air source 10 includes a motor 20 that drives a fan 22 to create a flow of air sourced from ambient air through the hot air source 10. The hot air source 10 also includes a heater 24 configured to heat the flow of air through the hot air source 10 to a desired temperature. Any suitable type of heater 24 can be used, such as those disclosed herein. The hot air source 10 can include an air inlet 26 that allows ambient air to enter an internal air conduit 28 of the heat source. 10. The internal air conduit 28 is connected to an air outlet port 30 opposite the air inlet 26. A breathing tube 14 and user interface 16 can be connected to the air outlet port 30 to receive a flow of hot air from the hot air source 10 and deliver it to the user via the breathing tube 14 and interface 16.

In some configurations, the hot air source 10 may include adjustable controls, which allows a user to adjust characteristics of the delivered air, such as temperature and/or humidity, and/or the length of the therapy session. In particular, the hot air source 10 can have a timer 40, which can determine the period of use. The timer 40 may be programmable to allow a user to select the period of use. The hot air source 10 can also include a pressure regulator 42 that adjusts a pressure of the flow of hot air delivered by the hot air source 10. The hot air source 10 can also include a flow regulator 44 that adjusts a flow rate of the flow of hot air delivered by the hot air source 10. The hot air source 10 can also include a temperature controller 46 that adjusts a temperature of the flow of hot air delivered by the hot air source 10. One or more of the pressure regulator 42, the flow regulator 44, and the temperature controller 46 can be user programmable, either directly or through selection of a particular mode that is stored in a memory and is operable by a controller of the hot air source 10.

The hot air source 10 can also include one or more input or output ports for communication with the internal air conduit 28 or other portions of the hot air source 10. For example, a first inlet port 50 can allow medication to be delivered to the user U by the hot air source 10. A second inlet port 52 can allow essential oils to be delivered to the user U by the hot air source 10. Additional inlet or outlet ports 54, 56, 58 can be provided for other uses.

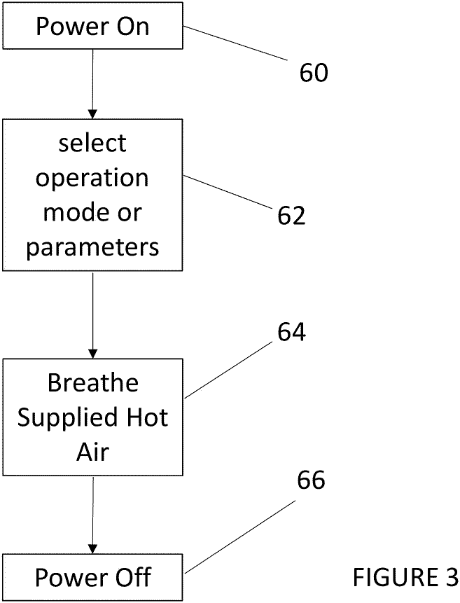

With reference to FIG. 3, in use, a user U can power on the hot air source 10, if necessary, as illustrated by block 60. The user U can then select a mode of operation from a plurality of available modes or can select particular characteristics of the delivered air, as described above, and as illustrated by block 62. The user U can then apply the desired user interface 16 (FIGS. 1 and 2) and then breathe the flow of hot air supplied by the hot air source 10, as illustrated by block 64. The conditioned, optionally pressurized, air would then be inhaled into the nose and or mouth and delivered to the lungs via the windpipe. The hood, mask, nasal cannula or other interface 16 can have an exhalation port incorporated in the system to allow the user to exhale. Once the session is complete, the user U can power off the hot air source 10 or the hot air source 10 can automatically power down, such as after a predetermined period without use, as illustrated by bock 66.

### Conclusion

It should be emphasized that many variations and modifications may be made to the herein-described embodiments, the elements of which are to be understood as being among other acceptable examples. All such modifications and variations are intended to be included herein within the scope of this disclosure and protected by the following claims. Moreover, any of the steps described herein can be performed simultaneously or in an order different from the steps as ordered herein. Moreover, as should be apparent, the features and attributes of the specific embodiments disclosed herein may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Moreover, the following terminology may have been used herein. The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” or “approximately” means that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “about 1 to about 3,” “about 2 to about 4” and “about 3 to about 5,” “1 to 3,” “2 to 4,” “3 to 5,” etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

What is claimed is:

1. A system for delivering hot air to a respiratory tract of a user, comprising:
a hot air source comprising a heater and a blower configured to produce a flow of hot air have a temperature of at least 150° F. and a relative humidity of less than 20%;
a user interface connected to the hot air source by a breathing tube, the user interface configured to receive the flow of hot air from the hot air source and deliver the flow of hot air to the respiratory tract of the user.

2. The system of claim 1, wherein the user interface is a nasal cannula, a mask, or a hood.

3. The system of claim 1, wherein the hot air source includes one or both of a pressure regulator or a flow regulator.

4. The system of claim 1, wherein the hot air source includes a temperature controller configured to adjust a temperature of the flow of hot air.

5. The system of claim 1, wherein the hot air source comprises one or more inlet or outlet ports configured to communicate with the flow of hot air.

6. The system of claim 5, wherein one of the one or more inlet ports is configured to permit medication to be delivered to the user via the flow of hot air.

7. A method of respiratory therapy, comprising:
delivering a flow of hot air to a respiratory tract of a user via a user interface;
wherein the flow of hot air has a temperature of at least 150° F. and a relative humidity of less than 20%.
